(19) United States
(12) Patent Application Publication
(10) Pub. No.: US 2026/0260068 A1
(43) Pub. Date: Sep. 3, 2026

(54) A COMPUTER-IMPLEMENTED METHOD FOR PROVIDING CARE

(71) Applicant: IESO DIGITAL HEALTH LIMITED, Cambridge (GB)

(72) Inventors: Mihai Valentin TABLAN, Cambridge (GB); Sabine Nicole BUCHHOLZ, Cambridge (GB); Ronan Patrick CUMMINS, Cambridge (GB); Justin Robert WASHTELL-BLAISE, Cambridge (GB)

(21) Appl. No.: 18/877,819

(22) PCT Filed: Jun. 23, 2023
(86) PCT No.: PCT/GB2023/051653

§ 371 Date: Dec. 20, 2024

(30) Foreign Application Priority Data
Jun. 24, 2022 (GB) 2209283.7

(51) Int. Cl. G06F 40/30; G06F 40/20; G16H 10/20; G16H 20/70; G16H 40/67; G16H 50/30

(52) U.S. Cl. CPC G06F 40/30; G06F 40/20; G16H 10/20; G16H 20/70; G16H 40/67; G16H 50/30

(57) ABSTRACT

A computer-implemented method is provided. The method comprising the steps of: receiving an input from a user; simultaneously analysing the input using a natural language understanding module of an active sub-dialogue unit and a natural language understanding module of at least one background sub-dialogue unit, wherein each natural language understanding module is configured to identify, if present within the input, at least one intent from a list of predetermined intents associated with the corresponding sub-dialogue unit; identifying each sub-dialogue unit comprising a natural language understanding module that has identified an intent; determining which one of the identified sub-dialogue units meets a predetermined criterion; selecting the sub-dialogue unit that meets the predetermined criterion; determining an output using a sub-dialogue planning module of the selected sub-dialogue unit, wherein the output is based, at least in part, on the at least one identified intent; and providing the output to the user using an output generation module of the selected sub-dialogue unit.

The present invention relates to computer-implemented methods for providing care and, more specifically, to computer-implemented methods for maintaining or improving a user's state of wellbeing.

Voice-driven computing and artificial intelligence is becoming more and more pervasive in our lives, supported by the presence and integration of such technology on our phones, appliances and in our cars. In coming years, talking to a computer, via text or voice, will increasingly be how many of us perform a growing number of activities. The awareness of an individual's state of well-being is also on the rise. Consequently, provisions for providing support, coaching, treatment and/or therapy are of interest.

These voice-driven computing systems are typically relatively uncomplex. The complexity of a bot running an interactive system may be measured in “turns”—i.e. the number of interactions between the bot and the user required to complete the activity. A bot that enables a user to, for example, check the weather forecast for a given location or confirm the timing of their next medication, may require between one and ten turns, for example.

In contrast, psychotherapy interactions are complex. In patient-therapist text-based cognitive behavioural therapy (CBT), a patient will typically spend around 6 hours in therapy sessions in which the CBT protocol is delivered. There will be, on average, around 50 “turns” per hour and therefore the system will need to handle of the order of several hundred turns. Other protocols, including specific forms of CBT protocols, may also be delivered. These protocols may be deemed ‘care protocols’.

In order to address this level of complexity in a care protocol, the protocol can be divided into a plurality of elements of care, each of which is delivered by a dedicated sub-dialogue unit. As such, the overall conversation, or dialogue, may be divided into a number of different stages, or sub-dialogues, wherein each stage, or sub-dialogue, is delivered by a separate sub-dialogue unit. One challenge that arises from the sub-division of the psychotherapy protocol into a plurality of sub-dialogue units each relating to an element of the care protocol, is that inputs and/or replies received from the user that do not relate to the currently active sub-dialogue unit must be managed appropriately. The conversational agent needs to be constantly aware of indications of risk throughout the delivery of the care protocol.

It is against this background that the present invention has arisen.

According to the present invention there is provided a computer-implemented method comprising: receiving an input from a user; simultaneously analysing the input using a natural language understanding module of an active sub-dialogue unit and a natural language understanding module of at least one background sub-dialogue unit, wherein each natural language understanding module is configured to identify, if present within the input, at least one intent from a list of predetermined intents associated with the corresponding sub-dialogue unit; identifying each sub-dialogue unit comprising a natural language understanding module that has identified an intent; determining which one of the identified sub-dialogue units meets a predetermined criterion; selecting the sub-dialogue unit that meets the predetermined criterion; determining an output using a sub-dialogue planning module of the selected sub-dialogue unit, wherein the output is based, at least in part, on the at least one identified intent; and providing the output to the user using an output generation module of the selected sub-dialogue unit.

In order to manage risk, a risk assessment sub-dialogue unit is provided which is configured to receive and analyse all inputs from the user even when the risk assessment sub-dialogue unit is not the active sub-dialogue unit or conversational agent within the system.

The provision of a separate risk assessment sub-dialogue unit avoids the requirement to replicate the risk programming into each separate sub-dialogue unit. This creates a more efficient system, but avoids duplication. However, in order for the risk assessment sub-dialogue unit to function effectively, an adjudicator is provided to enable the risk assessment sub-dialogue unit to interrupt a currently active sub-dialogue unit where an intent identifying risk is identified.

The risk assessment sub-dialogue unit can be called a background sub-dialogue unit in the sense that it listens to all inputs from the user even though it is not the active sub-dialogue unit. Other sub-dialogue units may also be provided that act as background sub-dialogue units. These may include, for example, frequently asked questions or even advertising.

The computer implemented method may be suitable for at least one of managing a digital conversation; managing risk; optimising a conversational agent; and/or providing psychotherapy.

The active sub-dialogue unit may have provided a previous output in response to a previous input.

If a natural language understanding module does not identify an intent from the list of predetermined intents associated with each sub-dialogue unit within the input, it may determine that no intent has been found.

The adjudicator may be used to select the sub-dialogue unit that meets the predetermined criterion.

The output generation module may be a natural language generation module. Alternatively, or in addition, the output generation module may be a multi-media output generation module.

The step of determining which one of the identified sub-dialogue units meets the predetermined criterion consists of one of: determining which one of the identified sub-dialogue units is the active sub-dialogue unit; assigning a predetermined priority value to each sub-dialogue unit and determining the identified sub-dialogue unit having the highest priority value; and determining a confidence value for each sub-dialogue unit, wherein the confidence value indicates how confident the corresponding natural language understanding module is in its identification of the intent, and determining the identified sub-dialogue unit having the highest confidence value.

For example, if the active sub-dialogue unit identifies an intent, it may be selected to determine the output. All of the background sub-dialogue units that identify an intent may be ignored. However, if the active sub-dialogue unit does not identify an intent, the background sub-dialogue units may be consulted, and if any one of them has identified an intent, it may be selected to determine the output.

This approach ensures more natural-flowing conversations, with fewer interruptions. As such, background sub-dialogue units may only be used to fill-in the gaps in the understanding capabilities of the active sub-dialogue unit.

In some embodiments, each sub-dialogue unit may be assigned a priority value. If multiple sub-dialogue units identify an intent, the one with the highest priority may be selected to determine the output. The multiple sub-dialogue units may comprise the active and/or background sub-dialogue units. This allows for flexibility in the design of the system, where certain sub-dialogue units get priority over the active sub-dialogue unit, while others do not.

Alternatively, in some embodiments, the natural language understanding modules of each sub-dialogue unit that identifies an intent may produce a confidence value associated with their prediction of a user intent. The confidence value may be implemented using statistical techniques. In such a setting, the sub-dialogue unit that is most confident in its interpretation of the user input may be selected to determine the output.

Alternatively, the step of determining which one of the identified sub-dialogue units meets the predetermined criterion may comprise: calculating an overall score for each identified sub-dialogue unit, wherein the overall score is calculated based on at least one of: determining which one of the identified sub-dialogue units is the active sub-dialogue unit; assigning a predetermined priority value to each sub-dialogue unit; and determining a confidence value for each sub-dialogue unit, wherein the confidence value indicates how confident the corresponding natural language understanding module is in its identification of the intent; and selecting the sub-dialogue unit having the highest overall score.

For example, if multiple sub-dialogue units identify an intent, one with the highest overall priority value may be selected to determine the output. However, if there are multiple sub-dialogue units with the same level of priority, then the one of those with the highest confidence value may be selected to determine the output. Similarly, the priority value assigned to each sub-dialogue unit may be used to select between two or more sub-dialogue units having the same confidence value.

Alternatively, or in addition, if a sub-dialogue unit has determined a previous output, it may not be eligible for determining another output during a given period of time. This can be used to limit the interventions from an advertising sub-dialogue unit, for example. In a clinical context, the priority value assigned to different sub-dialogue units may dynamically change depending on the symptoms, test results and/or stage reached in a user's treatment.

The priority values assigned to each sub-dialogue unit may be automatically determined and/or optimised by learning from user interactions with the system. This may be done by using explicit or implicit indicators of conversation success as an input to data-driven optimisation processes. Additionally, previously recorded conversations can be manually annotated for indicators of success. Situations where the selection of a particular sub-dialogue unit would have been beneficial can also be manually annotated by domain experts.

In some embodiments, there may be multiple previously active sub-dialogue units that have been ‘interrupted’ in favour of a selected background sub-dialogue unit. This may result in stacked interruptions. For example, a first active sub-dialogue unit may be ‘interrupted’ if a background sub-dialogue unit is selected to determine the output. The selected background sub-dialogue unit then becomes the second active sub-dialogue unit. This may lead to situations where the first active sub-dialogue unit has lost control in favour of a background sub-dialogue unit, which becomes the second active sub-dialogue unit, and, while the user interacts with the second active sub-dialogue unit, one of their inputs results in subsequent background sub-dialogue unit being selected to determine an output. The subsequent background sub-dialogue unit may then become the third active sub-dialogue unit. Therefore, the adjudicator is configured to keep track of the full conversation stack, noting all previous input, outputs, and/or replies within all previously active sub-dialogue units.

Once the conversation managed by an active sub-dialogue unit ends, the next user input needs to be analysed appropriately. Thus, if stacked interruptions are permitted, a decision needs to be taken on whether the next input goes back to the sub-dialogue unit at the top of the stack i.e. the one most recently interrupted, or to the originally active sub-dialogue unit thus closing all interrupted sub-dialogues simultaneously, or to an orchestrator. Therefore, calculating an overall score for each identified sub-dialogue unit may include deciding which sub-dialogue units were previously active and in which order.

At least one of the sub-dialogue units may be a risk sub-dialogue unit comprising a natural language understanding module configured to identify any intent indicating a risk.

Therefore, the list of predetermined intents associated with the risk natural language understanding module may each indicate a risk. The active sub-dialogue unit may be a risk sub-dialogue unit. Alternatively, at least one of the background sub-dialogue units may be a risk sub-dialogue unit.

The computer-implemented method may further comprise: assigning a predetermined priority value to each sub-dialogue unit, wherein the risk sub-dialogue unit is assigned the highest priority value; receiving an input from a user, wherein the input comprises an intent indicating a risk; identifying each sub-dialogue unit having a natural language understanding module that has identified an intent; determining that the risk sub-dialogue unit is the identified sub-dialogue unit having the highest priority value; selecting the risk sub-dialogue unit; determining an output using a sub-dialogue planning module of the risk sub-dialogue unit, wherein the output is based, at least in part, on the intent indicating a risk; and providing the output to the user using an output generation module of the risk sub-dialogue unit.

The output may be configured to confirm the presence of the intent indicating a risk within the input. Alternatively, or in addition, the output may seek to confirm the presence of the intent indicating a risk within the input. For example, the output may comprise a question relating to the presence of the intent indicating a risk.

The method may further comprise: receiving, in response to the output, a reply from the user confirming the presence of the intent indicating a risk.

The reply may be received by the natural language understanding module of the risk sub-dialogue unit. The risk natural language understanding module may be configured to identify, if present within the reply, at least one intent from a subsequent list of predetermined intents associated with the corresponding sub-dialogue unit. The subsequent list of predetermined intents may be a different list to the original list of predetermined intents associated with the corresponding sub-dialogue unit. Alternatively, it may be the same list.

In response to identifying at least one intent from the subsequent list of predetermined intents, the corresponding dialogue planning module may determine at least one subsequent output. The at least one subsequent output may be provided to the user using an output generation module.

Alternatively, the reply from the user may deny the presence of the intent indicating a risk. In this case, the reply may be treated as an input and the method restarted. Alternatively, an alternative intent within the original input may be analysed and responded to instead.

The method may further comprise: providing, using the output generation module of the risk sub-dialogue unit, at least one subsequent output to the user, wherein at least one subsequent output is configured to determine the severity of the risk associated with the intent indicating a risk.

The at least one subsequent output may be based, at least in part, on the input and/or reply. For example, the subsequent output may comprise at least one questions relating to the intent indicating a risk.

The method may further comprise: receiving at least one subsequent reply from the user; and estimating the severity of the risk based, at least in part, on the input, reply and/or at least one subsequent reply.

The severity of the risk may be estimated by identifying the category of risk into which the intent falls. Each category of risk has a different response appropriate to the risk expressed.

The method may further comprise taking an action, wherein the action is based, at least on part, on the estimated severity of the risk. For example, if the severity of the risk is low, the additional action may comprise logging the intent indicating a risk within a memory or sending a notification to the user's clinician. In addition, the output may comprise providing advice to the user. Conversely, if the severity of the risk is high, the action may comprise alerting emergency services. The user would also receive an appropriate output.

Each natural language understanding module may be further configured to identify, where present, at least one slot within the input; and wherein the corresponding output, if determined, is based, at least in part, on the at least one identified slot.

Determining the output based, at least in part, on the intent and/or slot associated with the input enables the exchange between the computer system and the user to be more conversational, thus improving user engagement.

The reply and/or subsequent reply may be the input for a subsequent iteration of the method. In other words, the input may be a previous reply.

Furthermore, according to the present invention there is provided a conversational agent comprising: an active sub-dialogue unit comprising: a natural language understanding module configured to receive an input from a user and, if present within the input, identify an intent from a list of predetermined intents associated with the active sub-dialogue unit; a sub-dialogue planning module configured to determine an output based, at least in part, on the identified intent from the list of predetermined intents associated with the active sub-dialogue unit; and an output generation module configured to provide the output, if determined, to the user; at least one background sub-dialogue unit comprising: a natural language understanding module configured to receive the input from the user and, if present within the input, identify an intent from a list of predetermined intents associated with the background sub-dialogue unit; a sub-dialogue planning module configured to determine an output based, at least in part, on the identified intent from the list of predetermined intents associated with the background sub-dialogue unit; and an output generation module configured to provide the output, if determined, to the user; and an adjudicator configured to: identify each sub-dialogue unit comprising a natural language understanding module that identifies an intent; determine which one of the identified sub-dialogue units meets a predetermined criterion; and select the sub-dialogue unit that meets the predetermined criterion such that only the selected sub-dialogue unit determines and provides an output to the user in response to each input.

In some embodiments, the input may be a reply, from the user, in response to a previous output.

The division of the care protocol into a number of different elements of care, each delivered by a dedicated sub-dialogue unit, makes each sub-dialogue unit more accurate and efficient as it works with a lower number of intents. However, the division into sub-dialogues, in turn, necessitates the management of the delivery of the care protocol as a whole. This management is provided by an orchestrator which introduces each sub-dialogue unit to the user and then steps in between each subsequent sub-dialogue unit to provide a bridge between the sub-dialogues, thus providing the user with a more engaging and natural conversation.

The orchestrator is configured as a sub-dialogue unit comprising a natural language understanding module, a dialogue planning module and a natural language generation module. In particular, the orchestrator natural language understanding module may be configured to receive an input and/or reply and determine at least one intent and, where present, at least one slot within the input and/or reply. The identified intent may be from a list of predetermined intents associated with the orchestrator sub-dialogue unit. The orchestrator dialogue planning module may be configured to determine an output based, at least in part, on the at least one intent and/or slot associated within the input and/or reply received by the orchestrator natural language understanding module. The orchestrator natural language generation module may be configured to provide the output determined by the orchestrator dialogue planning module to the user. The aim of the orchestrator is not to deliver an element of a care protocol, but to enhance the user's experience and increase or maintain their level of engagement with the psychotherapy. The conversational nature of the agent, delivered with the use of the orchestrator, gives the user an experience that is intended to mirror more closely interactions with a human therapist with the intention of keeping the level of engagement to a higher level than can typically be achieved by App based delivery.

The predetermined criterion may include a notification that the treatment protocol within the active sub-dialogue unit is finished and therefore control of the delivery of the care protocol can be handed back to the orchestrator.

The treatment protocol may be a series of steps for improving a user's state of wellbeing. The treatment protocol may be tailored for a specific user state, such as depression or anxiety. The treatment protocol may comprise a series of activities that may be provided to the user in the form of an output. Each activity may be provided in a specified order.

For example, a treatment protocol for Generalised Anxiety Disorder (GAD) may comprise socialising to the model, formulation, journaling worries (worry diary), worry classification, worry themes, worry time, progressive muscle group relaxation (PMGR) and/or planning for the future. A treatment protocol for depression may comprise socialising to the model, formulation, activity journaling, behavioural activation, cognitive restructuring, progressive muscle group relaxation (PMGR) and/or planning for the future.

A conversational agent may be required to maintain a conversation with its user that includes tens or even hundreds of inputs and outputs, spread throughout multiple days, or weeks. As the complexity of the designed interaction increases, that may have a detrimental effect on the performance of the system, and on its ease of development. A dialogue unit may be comprised of a natural language understanding (NLU) module, a dialogue planning (DP) module, and an output generation module. The output generation module may be a natural language generating (NLG) module. Dialogues that are more complex require the NLU module to be able to identify a greater number of intents and, where present, slots, which can lead to a drop in accuracy. This may pose a limit in complexity, beyond which the NLU accuracy is simply no longer sufficient for achieving a natural interaction. Longer lasting, and more complex conversations can lead to a DP module that is hard to design, implement, and maintain. This increases the costs of system development, and may again impose a limit on the complexity of the conversation, beyond which implementing a suitable DP module is no longer economically feasible.

One method to limit the complexity of a dialogue in a conversational agent, while increasing the complexity of the interaction model, is to break up a longer conversation into stages. Each stage can then be implemented as its own sub-dialogue, which has a lower complexity than would be required for the overall interaction. Crucially, each sub-dialogue has a significantly lower level of complexity compared to the overall conversation. Each sub-dialogue may be provided by a different sub-dialogue unit.

This approach is particularly well suited for structured conversations, such as would be encountered in the delivery of psychotherapy treatment protocol, or in the delivery of educational content. For example, in a psychotherapy context, the conversation may advance from one topic to another, moving from assessment of the patient symptoms, to understanding their goals in therapy, to psychoeducation, to the presentation of coping techniques, to experiential experiments, and so on. Each of these stages may be implemented as a self-contained and relatively simple sub-dialogue.

The output generation module of the active sub-dialogue unit may be a natural language generation module. Alternatively, the output generation module of the active sub-dialogue unit may be a multi-media generation module.

The output generation module of at least one background sub-dialogue unit may be a natural language generation module. Alternatively, the output generation module of at least one, or each, background sub-dialogue unit may be a multi-media generation module.

The natural language understanding module of at least one sub-dialogue unit may be configured to identify, where present, at least one slot within the input; and wherein the corresponding output, if determined, is based, at least in part, on the at least one identified slot.

As previously disclosed, determining the output based, at least in part, on the intent and/or slot associated with the input enables the exchange between the computer system and the user to be more conversational, thus improving user engagement.

The list of predetermined intents associated with the active sub-dialogue unit may be different from the list of predetermined intents associated with the background sub-dialogue unit.

However, at least one intent may be present on two or more different predetermined lists. As such, different predetermined lists may only comprise one difference. However, in some embodiments, different predetermined lists may comprise multiple differences or may be completely different. Also, in some embodiments the list of predetermined intents associated with the active sub-dialogue unit may be the same as the list of predetermined intents associated with the background sub-dialogue unit.

At least one sub-dialogue unit may be a risk sub-dialogue unit comprising: a natural language understanding module configured to receive the input from the user and, if present within the input, identify an intent indicating a risk; a sub-dialogue planning module configured to determine an output based, at least in part, on the identified intent indicating a risk; and an output generation module configured to provide the output to the user when facilitated by the adjudicator.

For example, in a clinical setting, therapists delivering care have a responsibility to monitor their patient for signs of risk to self or others. A similar responsibility may be assigned to the conversational agent. The watchful monitoring of user inputs for intents indicating a risk may be permanently present throughout a clinical conversation, regardless of the point currently reached in the interaction.

The risk sub-dialogue unit may be triggered by user inputs that include potential intents indicating a risk. Once triggered, the risk sub-dialogue unit may be selected to provide an output to the user.

The risk sub-dialogue unit may be further configured to take an action, and wherein the action is based, at least on part, on the identified risk.

For example, the risk sub-dialogue unit may be configured to confirm the presence of the risk and/or estimate the severity of the risk. The risk sub-dialogue unit may be further configured to enact one or more of a set of actions, depending on the outcomes of the discovery interaction. The actions may range from notifying the user's treating clinician, launching a crisis management procedure, involving clinical personnel, and/or calling out to the local emergency services, as appropriate.

The conversational agent may comprise a plurality of sub-dialogue units, one of which may be an orchestrator and one of which may be a background sub-dialogue unit. At any one time, only one sub-dialogue unit is active, in the sense that it is providing outputs to the user. The background sub-dialogue units are configured to receive each input.

The background sub-dialogue unit may be a risk sub-dialogue unit; a chitchat sub-dialogue unit; a conversation repair sub-dialogue unit; and FAQ sub-dialogue unit; an escalation sub-dialogue unit or an advertising sub-dialogue unit.

At least one of the sub-dialogue units may be a treatment sub-dialogue unit. Each treatment sub-dialogue unit may be used to deliver an element of care. Each element of care may comprise its own internal structure. For example, for “Defusion”, a typical element of care may include an introduction, a range of explanations and exercises through which the user can be guided. The element of care may further comprise elements of measurement (delivered as questionnaires or conversations) that can be used to track the progress that the user is making through the programme, and to measure the extent to which the required knowledge and understanding has been absorbed and internalised. A plurality of elements of care may be used to deliver a treatment protocol.

The orchestrator may be responsible for guiding the user from one sub-dialogue unit to another. Once an element of care managed by the active sub-dialogue unit is completed by the user, control may be handed back to the orchestrator. The orchestrator may be responsible for the conversations that the user has with the conversational agent until the next sub-dialogue unit becomes activated.

In addition, the orchestrator may be responsible for presenting the user with a unified experience, which hides the modular construction of the conversational agent. For technical reasons, it is preferable to limit the complexity of any one dialogue, therefore sub-dialogues are used to split the overall conversation into manageable smaller parts. However, it may not be desirable for the user to be presented with a fragmented user experience. Therefore, the orchestrator provides the conversational bridging that gives the illusion of a single long-lasting conversation.

The invention will now be further and more particularly described, by way of example only, with reference to the accompanying drawings; in which:

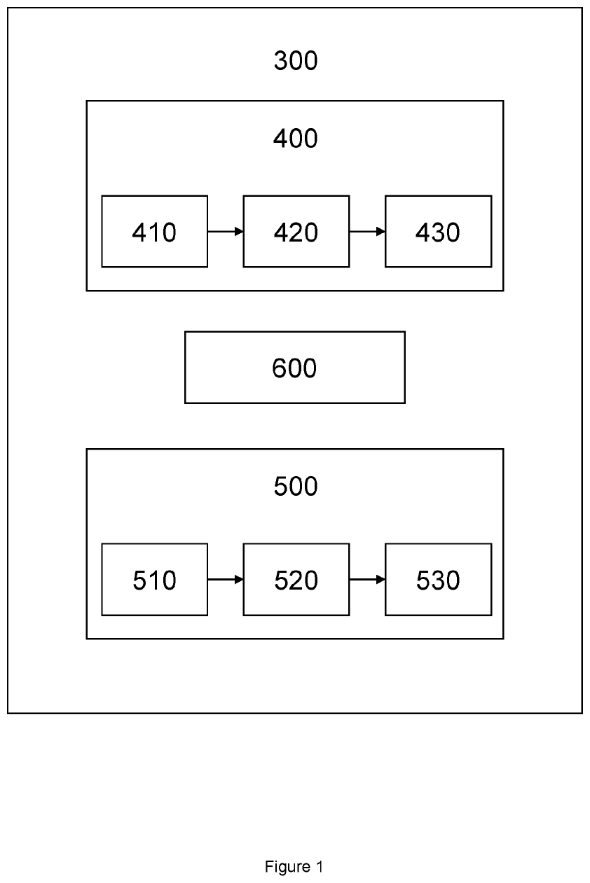

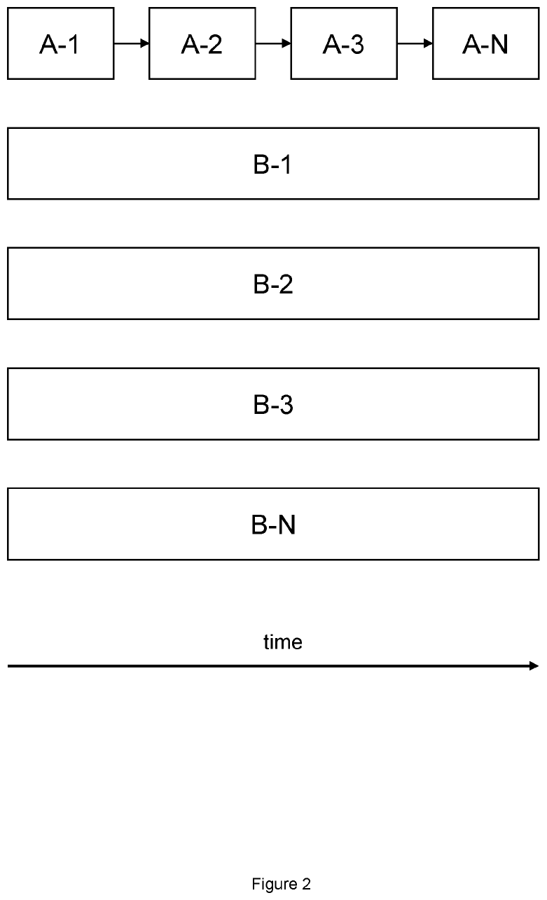

FIGS. 1 and 2 are a schematic of conversational agent according to some embodiments of the present invention;

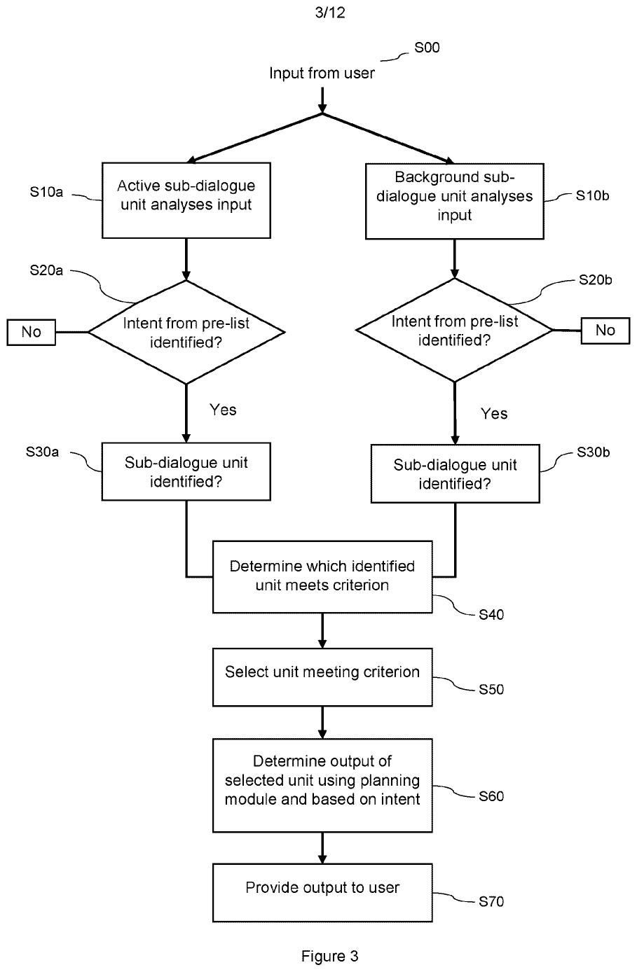

FIG. 3 is an overview flow diagram of a method according to the invention;

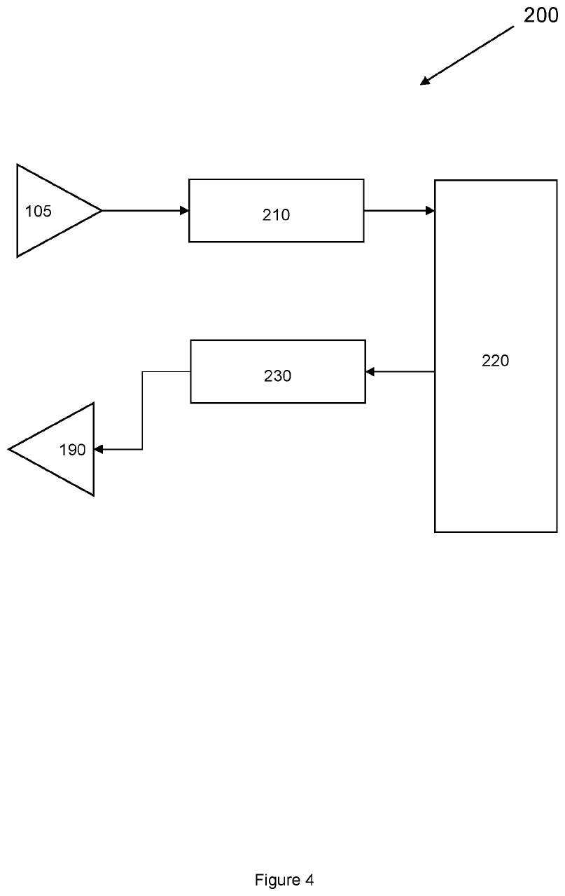

FIG. 4 shows a component of a computer-based system for maintaining or improving a user's state of wellbeing;

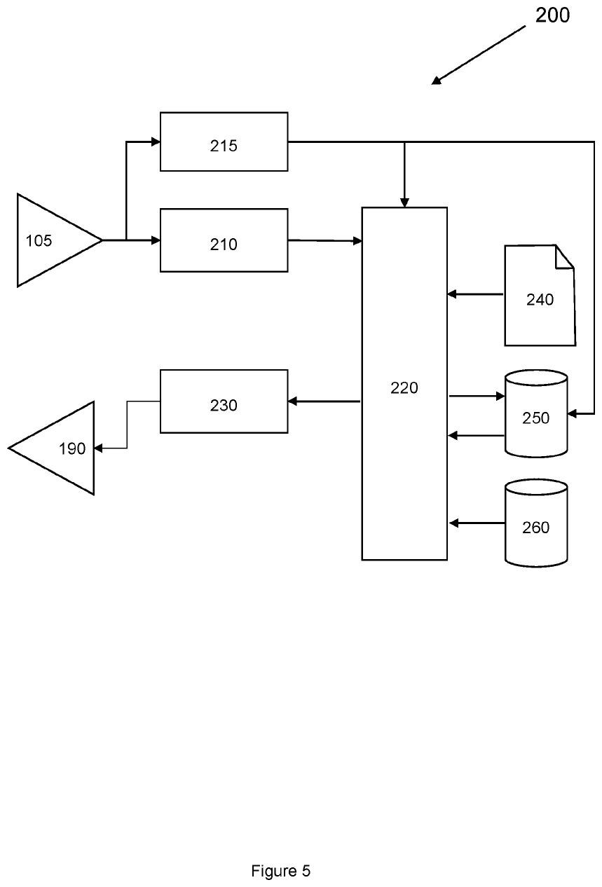

FIG. 5 shows the component in FIG. 4, including further modules;

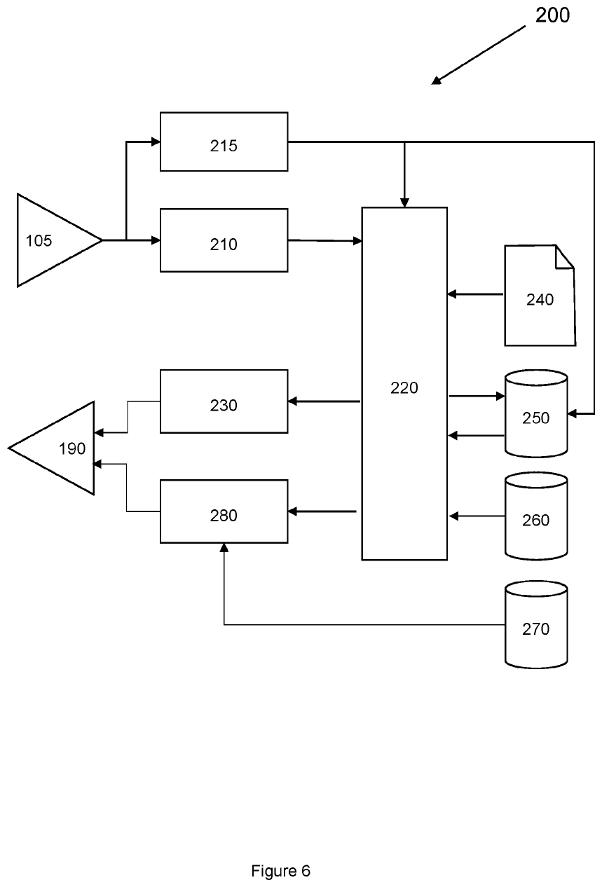

FIG. 6 shows the component in FIG. 5, including still further modules;

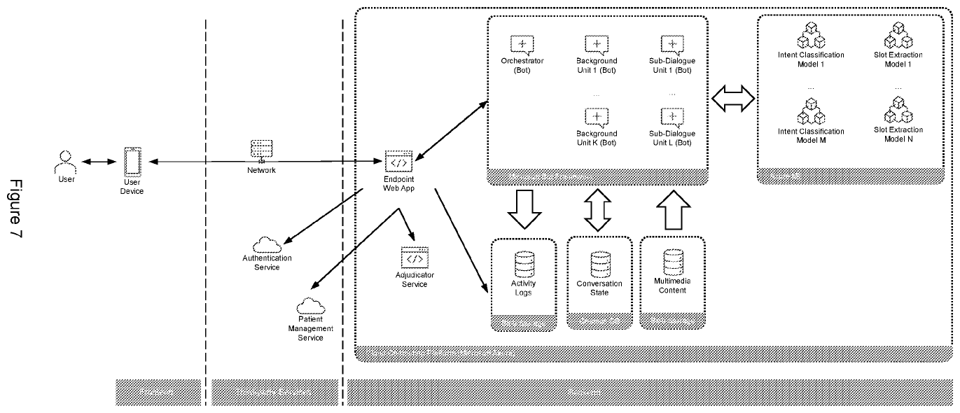

FIG. 7 shows an exemplary implementation;

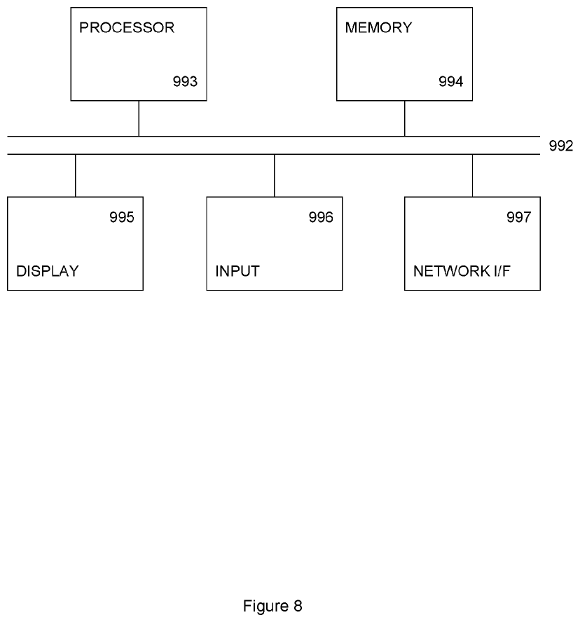

FIG. 8 is an overview hardware diagram;

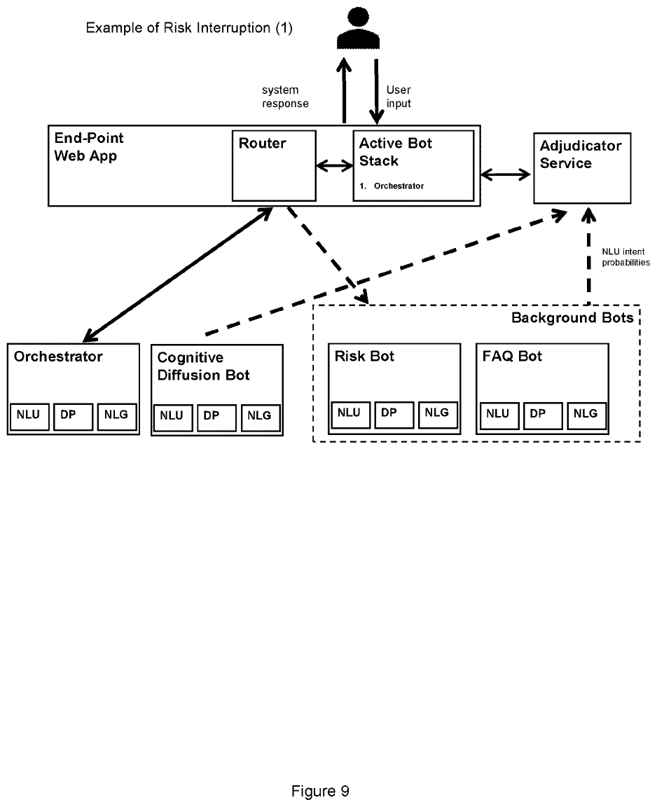

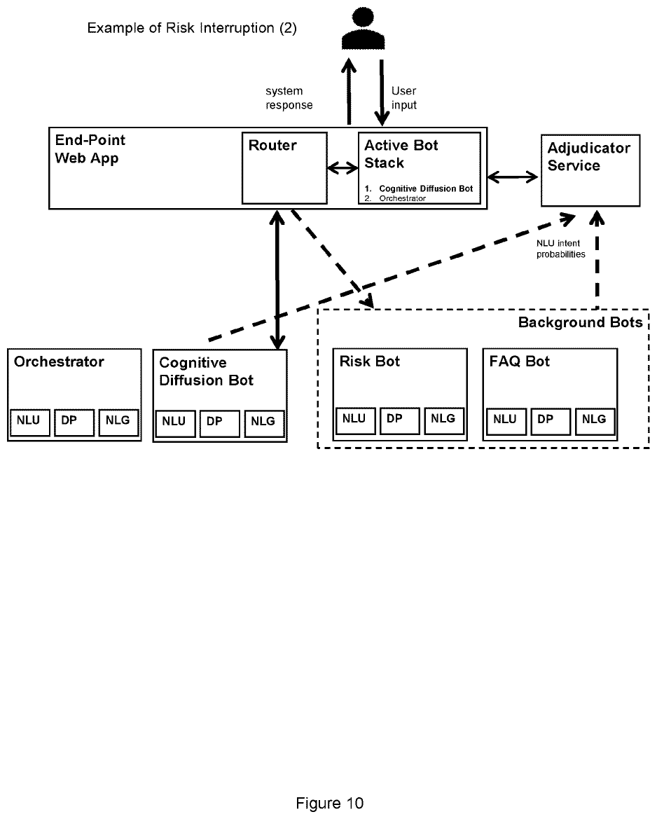

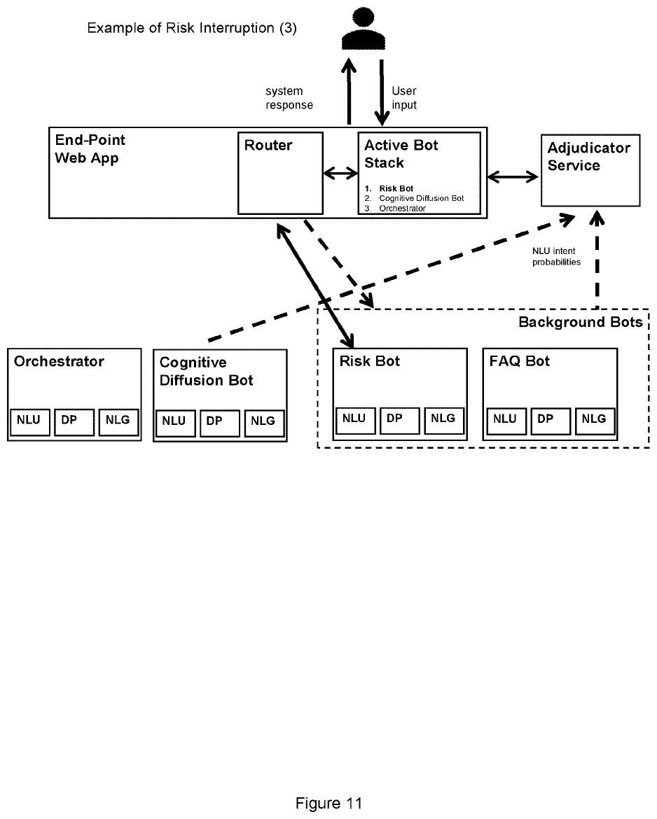

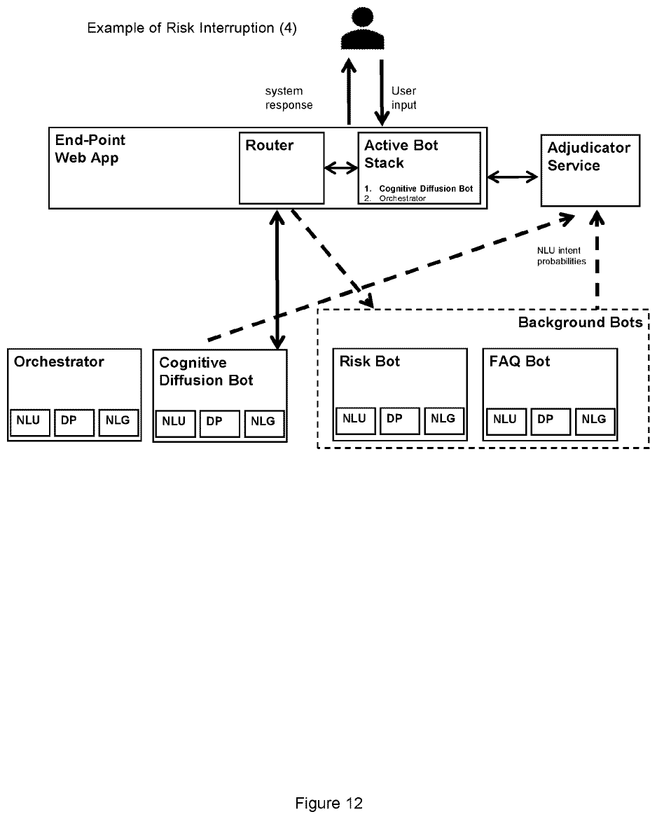

and FIGS. 9 to 12 show the logic flow between units for an exemplary conversation between the system and a user.

FIG. 1 shows a conversational agent 300 comprising an active sub-dialogue unit 400, a background sub-dialogue unit 500 and an adjudicator 600. FIG. 1 shows a single background sub-dialogue unit 500 for simplicity. However, any number of background sub-dialogue units may be present within the conversation agent 300.

The active sub-dialogue unit 400 comprises a natural language understanding module 410, a sub-dialogue planning module 420, and an output generation module 430. The active natural language understanding module 410 is configured to receive an input from a user and, if present, within the input, identify an intent from a list of predetermined intents associated with the active sub-dialogue unit 400. The predetermined list of intents for each sub-dialogue unit comprises between six and ten intents in most embodiments. It would be unusual for the number of intents on the predetermined list to exceed 20. The sub-dialogue unit will be more accurate and efficient when it works with a smaller number of intents. The active sub-dialogue planning module 420 is configured to determine an output based, at least in part, on the identified intent from the list of predetermined intents associated with the active sub-dialogue unit 400. The active output generation module 430 is configured to provide the output to the user.

Similarly, the background sub-dialogue unit 500 comprises a natural language understanding module 510, a sub-dialogue planning module 520, and an output generation module 530. The background natural language understanding module 510 is configured to receive an input from a user and, if present within the input, identify an intent from a list of predetermined intents associated with the background sub-dialogue unit 500. The background sub-dialogue planning module 520 is configured to determine an output based, at least in part, on the identified intent from the list of predetermined intents associated with the background sub-dialogue unit 500. The background output generation module 530 is configured to provide the output, where appropriate, to the user.

The adjudicator 600 is configured to identify each sub-dialogue unit comprising a natural language understanding module that identifies an intent; determine which one of the identified sub-dialogue units meets a predetermined criterion; and select the sub-dialogue unit that meets the predetermined criterion such that only the selected sub-dialogue unit determines and provides an output to the user in response to each input. One such criterion may be that the sub-dialogue unit has completed its delivery of its element of care and therefore control of the conversation should be handed back to the orchestrator.

FIG. 2 shows a conversational agent, in use, wherein the conversation agent comprises a plurality of sub-dialogue units, A-1 to A-N, and a plurality of background sub-dialogue units, B-1 to B-N. Any number of sub-dialogue units and/or background sub-dialogue units may be used. As shown, each sub-dialogue unit, A-1 to A-N, is configured to act in series. Therefore, a subsequent sub-dialogue unit A-2 is only able to gain control of the conversation when a previous sub-dialogue A-1 has finished. Therefore, no more than one sub-dialogue unit, A-1 to A-N, receives each input.

Conversely, each background sub-dialogue unit, B-1 to B-N, is configured to act in parallel with each other and the series of sub-dialogue units A-1 to A-N. Therefore, each background sub-dialogue units, B-1 to B-N, receives each input.

For example, a conversation may result in a plurality of sub-dialogue units being activated in series, with the orchestrator being activated briefly between each of the sub-dialogues that are configured to provide an element of care. Meanwhile, each background sub-dialogue unit receives each input from the user. However, a background sub-dialogue unit is only selected to determine and provide an output to the user if a predetermined criterion is met. If the predetermined criterion is met, the selected background sub-dialogue unit becomes the active sub-dialogue unit.

Various further aspects and embodiments of the present invention will be apparent to those skilled in the art in view of the present disclosure. “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments that are described. It will further be appreciated by those skilled in the art that although the invention has been described by way of example with reference to several embodiments, it is not limited to the disclosed embodiments and that alternative embodiments could be constructed without departing from the scope of the invention as defined in the appended claims.

FIG. 3 is an overview diagram of a method according to the invention. In step S00, user input is received, for example via a user interface on a user's device and an internet link to the computer which is carrying out the device. The input may be text or voice input, for example. In step S10a, the input is analysed by an active sub-dialogue unit (which is currently in dialogue with the user and producing outputs). Simultaneously in step S10b, the input is analysed by at least one background sub-dialogue unit, such as a risk assessment unit, FAQ unit or other unit that operates continuously even when it has not been selected as the active unit.

In S20a and S20b there is a step to identify, if present within the input, an intent from a list of intents associated with the unit. That is, each unit refers to an individual list of intents for its operation. If no intent is identified in any unit, then control may return to the orchestrator, or the currently active unit may continue to be active. In some embodiments, the active sub-dialogue unit may be programmed to always identify an intent. In this case, the “no” branch from S20a is not required. For example, if there is no specific intent in the list of intents identified by the sub-dialogue unit, it may default to a non-specific intent in the list of intents, such as “unclear intent”, for instance with a corresponding output to check what the user means, by asking the use to re-phrase the input.

In S30a and S30b there is an identification of each of the sub-dialogue units which has identified an intent. This identification may simply be an automatic status change.

In S40, the method determines which one of the sub-dialogue units meets a pre-determined criterion (as explained previously). In some simpler embodiments, the output may continue from the active sub-dialogue unit until the background sub-dialogue unit is required (or control is returned to the orchestrator) and the criterion for selection in S40 may be to choose the background sub-dialogue unit whenever it identifies an intent, or whenever it identifies an intent above a threshold probability. In this way the identification of an intent in the active sub-dialogue unit need not be considered in the selection of which sub-dialogue unit provides the output.

Only one unit is then selected in step S50, as the unit meeting the criterion. In step S60 the selected sub-dialogue unit determines an output, which is at least partially based on the input. In step S70, the output is provided to the user (for example from one or more servers carrying out the method to a UI on a user device).

FIG. 4 shows a component of a computer-based system for maintaining or improving a user's state of wellbeing, in the form of a sub-dialogue unit. The system 200 comprises a natural language understanding module 210, a dialogue planning module 220 and a natural language generation module 230. The natural language understanding module 210, a dialogue planning module 220 and a natural language generation module 230 form part of a conversational agent. More specifically, the natural language understanding module 210, dialogue planning module 220 and natural language generation module 230 form a sub-dialogue unit. A plurality of sub-dialogue units forms the basis for a conversational agent.

The natural language understanding module 210 is configured to receive an input 105 and determine at least one intent and, where present, at least one slot within the input. The dialogue planning module 220 is configured to determine an output 190 based, at least in part, on the intent and/or slot associated within the input. The natural language generation module 230 is configured to provide the output 190 to the user.

More specifically, the natural language understanding module 210 is configured to identify, if present within the input and/or reply, at least one intent from a list of predetermined intents associated with the sub-dialogue unit 200.

FIG. 5 shows the system shown in FIG. 4 further comprising a user engagement module 215, a treatment model module 240, a dialogue history module 250 and a patient data module 260.

The user engagement module 215 is configured to determine the user's engagement level. Its purpose is to estimate the likelihood that the user will perform the next expected action, such as providing a second input, for example. More specifically, the user engagement module reviews the input 105, all the previous inputs and some other metadata, such as time between responses, number of app activations over the last week, level of environmental noise at the user's current location. The user engagement module 215 then utilises a statistical method to make a determination of the user's current engagement level. In some embodiments, the statistical method is supplemented by a set of rules. Having determined the user's engagement level, the user engagement module 215 is then configured to provide the user engagement level to the dialogue planning module 220 for use in determining the output 190. The user engagement module 215 is also configured to provide the user engagement level to the dialogue history module 250.

The treatment model module 240 is configured to provide a computer-readable representation of a treatment protocol to the dialogue planning module 220, wherein the treatment protocol is a series of steps for improving a user's state of wellbeing. The treatment protocol is determined based, at least in part, on the input and is based on the obtained user data and/or data obtained during clinical assessment. The treatment model module 240 is configured to provide the treatment protocol to the dialogue planning module 220 for use in determining the output 190.

The dialogue history module 250 is configured to store previous inputs and previous outputs. The previous input is associated with a corresponding event, wherein the event comprises the previous input, a previous output and, where present, a second input. In some embodiments, the user engagement level is predicted, at least on part, based on a previous event. More preferably, the user engagement level is determined based on a plurality of previous events, wherein the plurality of previous events correspond to inputs received from a plurality of previous user. For example, the dialogue history module 250 may store previous inputs, output and/or events corresponding to tens, hundreds, thousands or millions of previous users.

The user data module 260 is configured to store information about the user. For example, the user data module may comprise the user's name, age, contact details, family members, symptoms, intensity of symptoms, triggers and/or frequency of input, to name a few. However, the previous list is non-exhaustive. Any information regarding the user may be stored. In some embodiments, the data stored in the user data module 260 is used, at least in part, to determine the output. For example, the output for a younger user may comprise a game, whereas the corresponding output for an older user may comprise a task. However, the output may be adapted in any suitable or appropriate way based on the user's data.

FIG. 6 shows the system shown in FIG. 5 further comprising a content module 270 and a content delivery module 280.

The content module 270 is configured to store audio and/or visual data. Moreover, the content module 270 is configured to store predefined data for providing to the user. The predefined data may comprise videos, games and/or documents. In some embodiments, the content module 270 also comprises a plurality of phrases, words or stylistic features that correspond to a given human care provider. Consequently, the output 190 can be adapted to replicate the response of a given human care provider.

The content delivery module 280 is configured to receive data from the content module 270 and generate an output 190. Therefore, in some embodiments, the output 190 comprises data from the natural language generation module 230 and the content delivery module 280.

For example, a user's input may comprise “good morning”, to which the computer may assign the intent ‘greeting’, with no slots. Alternatively, or in addition, a user's input may comprise “I feel like I am letting everyone down by not being able to go out”, to which the computer may assign the intent ‘inform_thought’, with the slot value ‘thought_self_blame’.

Table 1, below, shows a series of potential inputs and their corresponding intents and, where present, slots. The inputs shown in Table 1 may be received from a user subjected to “Vicious cycle” activity, wherein “Vicious cycle” activity is a form of CBT formulation. The purpose of the “Vicious cycle” activity is to explain the relationships between thoughts, physical sensations, emotions, and behaviours. The key message conveyed to a user is that each of their thoughts, physical sensations, emotions, and behaviours can reinforce each other in a vicious cycle which leads to increased and sustained anxiety. The present invention may be used to assist a user with breaking this cycle.

TABLE 1
example user inputs and their corresponding intents and, where present, slots.

| Input | Intent | Slots | Description |
|---|---|---|---|
| Yes | affirm | None | The user agrees or |
| Of course | | | confirms. |
| cool | | | |
| no | deny | None | The user disagrees |
| nope | | | |
| none | | | |
| definitely not | | | |
| Goodbye | goodbye | None | The user greets the |
| Good night | | | system at the end of a |
| bye | | | conversation. |
| hi | greet | None | The user greets the |
| hello | | | system at the start of a |
| hi there | | | conversation |
| I feel like I am letting | inform_thought | thought_self_blame | User describes a thought |
| everyone down by not being | | | they had |
| able to go out. | | | |
| I felt completely helpless and | inform_thought | thought_not_being_in_control | User describes a thought |
| lost and had nobody to make | | | they had |
| decision for me. | | | |
| The emotion going through | inform_thought | thought_inadequacy | User describes a thought |
| my mind at the time is failure | | | they had |
| and if someone says that | | | |
| about me it must be true. | | | |
| my heart would be faster and | inform_sensation | sensation_heart_palpitations | User describes a physical |
| I'd feel sick and uneasy | | sensation_stomach_problems | sensation |
| I just notice that I'm really | inform_sensation | sensation_muscle_tension | User describes a physical |
| tense | | | sensation |
| When it's bad I get a racing | inform_sensation | sensation_heart_palpitations | User describes a physical |
| heart | | | sensation |
| Fear and anxiety about what | inform_emotion | emotion_fear | User describes an emotion |
| might happen | | emotion_anxiety | they felt |
| Which in turn leads me to | inform_emotion | emotion_sadness | User describes an emotion |
| feel terrible | | | they felt |
| I felt disheartened and | inform_emotion | emotion_sadness | User describes an emotion |
| maybe a bit let down | | | they felt |
| I often go onto my phone and | inform_behaviour | behaviour_distraction | User describes a |
| check social media | | | behaviour they engaged in |
| I thought about skipping | inform_behaviour | behaviour_avoidance | User describes a |
| work and going somewhere | | | behaviour they engaged in |
| so I could be alone | | | |
| I was a little short tempered | inform_behaviour | behaviour_aggression | User describes a |
| with my daughter. | | | behaviour they engaged in |

Alternatively, or in addition, a slot may be: thought_self_conscious; thought_catastrophising; thought_others_wellbeing; thought_own_wellbeing; thought_getting_things_done; sensation_difficulty_concentrating; sensation_shaking; sensation_chest_problems; sensation_sweating; sensation_tiredness; sensation_flushes; sensation_breathing_difficulties; emotion_anger; emotion_guilt; emotion_frustration; behaviour_checking; behaviour_overplanning; behaviour_suppression; behaviour_reassurance; and/or behaviour_medication, for example.

However, various further aspects and embodiments of the present invention will be apparent to those skilled in the art in view of the present disclosure. Accordingly, the provided examples are exemplary only and are not limiting.

### Exemplary Implementation

An example implementation is illustrated in FIG. 7, with a set of technology choices for the elements described above.

In this implementation, an overall system which carries out the methods described and which provides the conversational agent described comprises two main parts: a frontend and a backend. The user interacts with the frontend to receive the outputs of the conversational agent and to allow input. For these purposes the frontend may use a user interface, such as a touch screen and/or audio input and output. The backend comprises all the services that provide the core functionality. Additionally, a small set of externally supplied third-party services may be used to provide functionality such as authentication, and patient data management.

The frontend is resident on a user device, typically a mobile phone, but potentially also a personal digital assistant, personal computer, laptop computer or other computing device. The user device communicates with the backend via the internet computer network, using standard communication protocols, such as TCP/IP, HTTP, and REST.

The backend comprises a collection of software services hosted within a cloud computing platform, such as Microsoft Azure (of course implementation on a single fixed server is also a possibility the skilled person will be aware of).

All communication with the backend is routed via an endpoint web app, which coordinates the activities of the various backend services in a way that provides the desired behaviour for the user, as detailed next.

The endpoint web app accesses the authentication service to validate the user's credentials and confirm their permissions to access the conversational agent. The authentication service can be implemented using existing off-the-shelf technology, such as Azure Active Directory, or other third-party solutions, like Auth0.

After authenticating the user, the endpoint web app may connect to a patient management service to log the user's access of the conversational agent, which may be required for administrative purposes, such as billing. Depending on the clinical pathway set in place, the user may be required to complete a series of tasks, such as filling in a range of clinical questionnaires. The results of these may also be stored via the patient management service.

After completing the administrative tasks required when access to the system is initiated, the user is given access to the functionality of the conversational agent. The endpoint web app does that by routing frontend requests to the orchestrator previously described. The orchestrator welcomes the user to the new session, retrieves any pre-existing conversation state, and hands over control of the conversation to the appropriate sub-dialogue unit.

All the elements marked “Bot” in FIG. 7, including the orchestrator, are implementations of sub-dialogue units in application-level software to execute tasks. These may be implemented using the Microsoft Bot Framework, or other third-party solution, such as RASA. These bot implementations provide, for example, the functionality of the dialogue planner component (sub-dialogue planning module), and natural language generation component (natural language generation module) in the conceptual framework described by this invention.

Bots also require an implementation for the natural language understanding functionality, which comprises a set of intent classification models, and slot extraction models. Each bot may use zero, one, or more intent classification models, and slot extraction models. Each intent classification model, and slot extraction model may be used by one, or shared between several bots. For example, the intent classification model that recognises agreement (i.e. phrases like ‘yes’, ‘of course’, ‘for sure’, ‘makes sense’, etc.) may be used in multiple places, and by multiple bots, whereas more specialised intent classification models may only be used in one place, by a single bot. Consequently, there is a many-to-many mapping of bots to models, and there is no direct relationship between the number of bots, the number of intent classification models, and the number of slot extraction models.

Intent classification models, and slot extraction models are machine learning models and may be implemented as custom-built components hosted within the Azure ML service. Alternatively, they could be based on Azure Cognitive Services for Language Understanding, or be suitably configured large language models such as those offered by the Azure OpenAI Service.

In order to allow continuation of previously interrupted conversations, bots are able to persistently store the conversation state for each user. This may be implemented using an Azure Cosmos DB datastore, or some other similar solution.

If multimedia content is part of the designed user experience, it can be stored within Azure Blob Storage and made available to bots that way. Bots have the option to retrieve such content from storage and return it as part of their response to the user's request.

Along with routing requests to the orchestrator, or the currently active sub-dialogue unit, the endpoint web app also sends all requests to all of the background units. If one or more of the background units identifies an intent within the latest user utterance, the endpoint web app needs to decide whether to cede control of the conversation to one of the background units and which one of them. To make that determination, the endpoint web app uses the adjudicator service, which may itself be implemented as a web app providing a REST API. The adjudicator implements the decision logic that takes into account the relative priority of all the bots that are able to provide a response, their confidence (i.e. detection probability) for the intent they have each identified, and a set of rules implementing other relevant business logic.

For monitoring and compliance reasons it may be necessary to maintain comprehensive activity logs that keep a record of all the interactions between the user and the system, and all the decisions made by the system. One way to implement these is by storing a record of all the system events, each event being represented as a snippet of XML or JSON content. The totality of all these event representations is then collected and persistently stored in Azure Blob Storage, or some other storage solution.

### Hardware

FIG. 8 is a block diagram of a computing device, such as a data storage server, which embodies the present invention, and which may be used to implement a method of an embodiment of the invention. The computing device comprises a processor 993, and memory, 994. The computing device also includes a network interface 997 for communication with other computing devices, for example with other computing devices implementing invention embodiments or parts thereof.

For example, an embodiment may be composed of a network of such computing devices, providing the cloud computing platform with the components shown in FIG. 7. Of course, the functionality could also be provided on a single server. Optionally, the computing device also includes one or more input mechanisms such as keyboard and mouse 996, and a display unit such as one or more monitors 995. The components are connectable to one another via a bus 992.

The memory 994 may include a computer readable medium, which term may refer to a single medium or multiple media (e.g., a centralized or distributed database and/or associated caches and servers) configured to carry computer-executable instructions or have data structures stored thereon. Computer-executable instructions may include, for example, instructions and data accessible by and causing a general-purpose computer, special purpose computer, or special purpose processing device (e.g., one or more processors) to perform one or more functions or operations. Thus, the term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device and execute processing operations, for example executing code stored in the memory to implement one, some or all of the various different functions of the bot framework, modelling, storage, adjudicator service and endpoint web app described here and in the claims. The memory 994 stores data being read and written by the processor 993. As referred to herein, a processor may include one or more general-purpose processing devices such as a microprocessor, central processing unit, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also include one or more special-purpose processing devices such as an application specific integrated circuit (ASIC), a field programmable gate array (FPGA), a digital signal processor (DSP), network processor, or the like. In one or more embodiments, a processor is configured to execute instructions for performing the operations and steps discussed herein.

The display unit 997 may display a representation of data stored by the computing device and may also display a cursor and dialog boxes and screens enabling interaction between a user (administrator) and the programs and data stored on the computing device. The input mechanisms 996 may enable a user to input data and instructions to the computing device.

The network interface (network I/F) 997 may be connected to a network, such as the Internet, and is connectable to other such computing devices, such as a user (patient) device via the network. The network I/F 997 may control data input/output from/to other apparatus via the network. Other peripheral devices such as microphone, speakers, printer, power supply unit, fan, case, scanner, trackerball etc may be included in the computing device.

The conversational agent 300 of FIG. 1 may be a processor 993 (or plurality thereof) executing processing instructions (a program) stored on a memory 994 and exchanging data via a network I/F 997. In particular, the processor 993 may execute processing instructions to act as a currently active-sub-dialogue unit and thus to receive, via the network I/F, input from a user (patient) and identify an intent (if present) and determine a corresponding output and then to output it to the user via the network I/F for display on the user device. Additionally or alternatively, the processor 993 may execute processing instructions to act as a background sub-dialogue unit, and thus to receive, via the network I/F, the same input from a user (patient) and identify an intent (if present) and determine a corresponding output and then to output it to the user via the network I/F for display on the user device. Furthermore, the processor 993 may execute processing instructions to act as an adjudicator, and thus to identify any sub-dialogue units (out of the active sub-dialogue unit and the background sub-dialogue units) which identifies an intent, determine which one meets a pre-determined criterion (such as highest priority or highest confidence, or some function of these factors taken together) and select that sub-dialogue unit, so that only the selected sub-dialogue unit provides its output to the user. Of course, if the cloud arrangement of FIG. 7 is used, then the physical location of the processing is not fixed: a different server (and thus processor) may provide the adjudicator and the endpoint web app. The sub-dialogue units may be hosted on different servers and call intent classification models and slot extraction models which may again be hosted elsewhere.

Methods embodying the present invention may be carried out on a computing device such as that illustrated in FIG. 8. Such a computing device need not have every component illustrated in FIG. 8, and may be composed of a subset of those components. A method embodying the present invention may be carried out by a computing device in communication with one or more data storage servers via a network. The computing device may be a data storage itself storing the resultant data in the invention.

FIG. 8 may also be used to represent the user device. In this case the user device communicates via the network I/F with the endpoint web app using standard communication protocols, such as TCP/IP, HTTP, and REST. Display 995 displays a user interface controlled by the conversational agent and providing the frontend introduced above. Input 996, in the form of a touchscreen or screen and keyboard and/or voice, is used for user input. The user interface can be embodied as a user app shown on the display and optionally connected to the audio of the user device for voice input and audio output. Some local storage, for example of a UserID and/or settings, can be provided by memory 994, and processor 993 can carry out background functions, but the core functionality is preferably implemented remotely from the user device for example on the cloud as described above.

### Worked Example—Logical Structure

FIGS. 9 to 12 illustrate a worked example and use the same basic diagram structure to explain interactions between the user and the system in a digital therapy conversation. The endpoint web app coordinates the activities of a number of services and routes user inputs to the active bot which is one of the orchestrator, a sub-dialogue unit, or a background bot (these background bots are also sub-dialogue units, as explained above, but are “always on”). The active bot is shown in FIG. 9 as the orchestrator). FIG. 9 shows a single exemplary sub-dialogue unit, as the cognitive diffusion bot, but the reader will appreciate that there are many other sub-dialogue units that may become active when selected by the orchestrator, as explained above.

The active bot stack manages the interruption of bots such that the any incoming user input is always routed to the active bot (i.e., the bot at the top of the stack).

The orchestrator is always the first bot placed on the stack and is responsible for welcoming the user. During the programme as sub-dialogue units (whether background bots or “normal” sub-dialogue units acting in series) become active, they get added to top of the stack. When they are completed, they get removed from the Active Bot Stack whereby the orchestrator will again become the active bot and introduce the next sub-dialogue unit.

The router in the end-point web app is responsible for sending the user input to the currently active bot and to the background bots. The user input is sent to the background bot's and to the active bot's NLU modules/components (such as the intent classification and slot extraction models shown in FIG. 7) and these return intent probabilities determining the degree to which the incoming input can be serviced by the active bot and each of the specific background bots (e.g. risk, FAQ). If the adjudicator service determines that a user input is best responded to by a background bot, it pushes the background bot (e.g. risk) to the top of the active bot stack so that the appropriate bot can handle the incoming message.

At times, multiple background bots may be able to respond to the user request and would compete with the active-bot to respond to the user input. This is indicated by the background bot NLU intents being above a specific threshold probability. This threshold can be different for different background bots, but for the following example we consider the simple case where this threshold is 0.5 for all background bots. Consider an input that had both risk and FAQ characteristics and assume that each relevant background bot was identified as being able to service this input (e.g. probability(risk-bot)=0.75, probability(FAQ-bot)=0.8). The adjudicator service determines which background bot to make the active bot by placing the highest priority bot that can service the input on the top of the active bot stack. Let's also assume that the priorities of the bots are 2, 1, and 1 for the risk-bot, the FAQ-bot and active-bot respectively. This adjudicator service considers the active bot and all background bots whose intent probability is above their specific threshold, and selects the bot with the highest predefined priority to service the intent (e.g. in this example checking risk has a priority of 2 while all other bots have a lower priority). If two bots have the same priority, the adjudicator will select the bot with the higher probability of servicing the user inputs.

### Specific Worked Example—Patient and DTx Conversation

### Context:

The user has started conversing with the orchestrator. At any point in time, the unfinished bots are in the active bot stack with an incoming user input being routed to the active bot and all background Bots.

Sample conversation with numbering corresponding to FIGS. 9, 10, 11 and 12.

| Figure | Bot or User | User input / System output | Description |
|---|---|---|---|
| 9 | [orchestrator] | Welcome to the programme. | The user is currently conversing |
| | | Let's begin with a cognitive | with the orchestrator bot. |
| | | diffusion exercise. Are you | |
| | | happy to continue? | |
| 9 | [user] | Yeah, sure! | The cognitive diffusion bot is |
| | | | pushed onto the top of the |
| | | | active bot stack becoming the |
| | | | active bot |
| 10 | [cognitive | What do you do when you have | The user is currently conversing |
| | diffusion bot] | thoughts in difficult situation? | with the cognitive diffusion bot. |
| 10 | [user] | What do you mean? | No background bots NLU intent |
| | | | probabilities are high enough to |
| | | | indicate they should interrupt, |
| | | | so conversation continues with |
| | | | the active bot. |
| 10 | [cognitive | Maybe I can put it another way. | cognitive diffusion bot |
| | diffusion bot] | Do you notice your behaviour | responds. |
| | | change? | |
| 10 | [user] | You mean like if my behaviours | The FAQ and risk bots NLUs |
| | | are related thoughts of harming | return with values higher than a |
| | | myself? | threshold suggesting they could |
| | | | service this request. The |
| | | | adjudicator service with access |
| | | | to the background bot priority |
| | | | determines that the risk bot |
| | | | should service this request and |
| | | | pushes it to the top of the active |
| | | | bot stack making it the active |
| | | | bot. |
| 11 | [risk bot] | I'd just like to check if have any | The risk bot responds. |
| | | thoughts of self-harm? | |
| 11 | [user] | No, not at all. | The Risk Bot ends and is pushed |
| | | | off the active bot stack making |
| | | | the cognitive diffusion bot the |
| | | | active bot. |
| 12 | [cognitive | Thanks. So, can you think of | cognitive diffusion bot |
| | diffusion bot] | alternative or more useful | responds. |
| | | behaviours in those situations? | |
| . . . | . . . | . . . | . . . |

1. A computer-implemented method comprising:
receiving an input from a user;
simultaneously analysing the input using a natural language understanding module of an active sub-dialogue unit and a natural language understanding module of at least one background sub-dialogue unit, wherein each natural language understanding module is configured to identify, if present within the input, at least one intent from a list of predetermined intents associated with the corresponding sub-dialogue unit;
identifying each sub-dialogue unit comprising a natural language understanding module that has identified an intent;
determining which one of the identified sub-dialogue units meets a predetermined criterion;
selecting the sub-dialogue unit that meets the predetermined criterion;
determining an output using a sub-dialogue planning module of the selected sub-dialogue unit, wherein the output is based, at least in part, on the at least one identified intent; and
providing the output to the user using an output generation module of the selected sub-dialogue unit.

2. The computer-implemented method according to claim 1, wherein determining which one of the identified sub-dialogue units meets the predetermined criterion consists of one of:
determining which one of the identified sub-dialogue units is the active sub-dialogue unit;
assigning a predetermined priority value to each sub-dialogue unit and determining the identified sub-dialogue unit having the highest priority value; and
determining a confidence value for each sub-dialogue unit, wherein the confidence value indicates how confident the corresponding natural language understanding module is in its identification of the intent, and determining the identified sub-dialogue unit having the highest confidence value.

3. The computer-implemented method according to claim 1, wherein determining which one of the identified sub-dialogue units meets the predetermined criterion comprises:
calculating an overall score for each identified sub-dialogue unit, wherein the overall score is calculated based on at least one of:
determining which one of the identified sub-dialogue units is the active sub-dialogue unit;
assigning a predetermined priority value to each sub-dialogue unit; and
determining a confidence value for each sub-dialogue unit, wherein the confidence value indicates how confident the corresponding natural language understanding module is in its identification of the intent; and
selecting the sub-dialogue unit having the highest overall score.

4. The computer-implemented method according to claim 1, wherein at least one sub-dialogue unit is a risk sub-dialogue unit comprising a natural language understanding module configured to identify an intent indicating a risk.

5. The computer-implemented method according to claim 4, further comprising:
assigning a predetermined priority value to each sub-dialogue unit, wherein the risk sub-dialogue unit is assigned the highest priority value;
receiving an input from a user, wherein the input comprises an intent indicating a risk;
identifying each sub-dialogue unit having a natural language understanding module that has identified an intent;
determining that the risk sub-dialogue unit is the identified sub-dialogue unit having the highest priority value;
selecting the risk sub-dialogue unit;
determining an output using a sub-dialogue planning module of the risk sub-dialogue unit, wherein the output is based, at least in part, on the intent indicating a risk; and
providing the output to the user using an output generation module of the risk sub-dialogue unit.

6. The computer-implemented method according to claim 5, wherein the output is configured to confirm the presence of the intent indicating a risk within the input.

7. The method according to claim 6, further comprising:
receiving, in response to the output, a reply from the user confirming the presence of the intent indicating a risk.

8. The method according to claim 7, further comprising:
providing, using the output generation module of the risk sub-dialogue unit, at least one subsequent output to the user, wherein at least one subsequent output is configured to determine the severity of the risk associated with the intent indicating a risk.

9. The method according to claim 8, further comprising:
receiving at least one subsequent reply from the user; and
estimating the severity of the risk based, at least in part, on the input, reply and/or at least one subsequent reply.

10. The method according to claim 9, further comprising taking an action, wherein the action is based, at least on part, on the estimated severity of the risk.

11. The method according to claim 1, wherein each natural language understanding module is further configured to identify, where present, at least one slot within the input; and wherein the corresponding output, if determined, is based, at least in part, on the at least one identified slot.

12. A conversational agent, the conversational agent comprising:
an active sub-dialogue unit comprising:
a natural language understanding module configured to receive an input from a user and, if present within the input, identify an intent from a list of predetermined intents associated with the active sub-dialogue unit;
a sub-dialogue planning module configured to determine an output based, at least in part, on the identified intent from the list of predetermined intents associated with the active sub-dialogue unit; and
an output generation module configured to provide the output, if determined, to the user;
at least one background sub-dialogue unit comprising:
a natural language understanding module configured to receive the input from the user and, if present within the input, identify an intent from a list of predetermined intents associated with the background sub-dialogue unit;
a sub-dialogue planning module configured to determine an output based, at least in part, on the identified intent from the list of predetermined intents associated with the background sub-dialogue unit; and
an output generation module configured to provide the output, if determined, to the user; and
an adjudicator configured to:
identify each sub-dialogue unit comprising a natural language understanding module that identifies an intent;
determine which one of the identified sub-dialogue units meets a predetermined criterion; and
select the sub-dialogue unit that meets the predetermined criterion such that only the selected sub-dialogue unit determines and provides an output to the user in response to each input.

13. The conversational agent according to claim 12, wherein the output generation module of the active sub-dialogue unit is a natural language generation module.

14. The conversational agent according to claim 12, wherein the output generation module of at least one background sub-dialogue unit is a natural language generation module.

15. The conversational agent according to claim 12, wherein the natural language understanding module of at least one sub-dialogue unit is configured to identify, where present, at least one slot within the input; and wherein the corresponding output, if determined, is based, at least in part, on the at least one identified slot.

16. The conversational agent according to claim 12, wherein the list of predetermined intents associated with the active sub-dialogue unit is different from the list of predetermined intents associated with the background sub-dialogue unit.

17. The conversational agent according to claim 12, wherein at least one sub-dialogue unit is a risk sub-dialogue unit comprising:
a natural language understanding module configured to receive the input from the user and, if present within the input, identify an intent indicating a risk;
a sub-dialogue planning module configured to determine an output based, at least in part, on the identified intent indicating a risk; and
an output generation module configured to provide the output to the user as facilitated by the adjudicator.

18. The conversational agent according to claim 17, wherein the risk sub-dialogue unit is further configured to take an action, and wherein the action is based, at least on part, on the identified risk.

19. The conversational agent according to claim 12, comprising at least one background sub-dialogue unit which is configured to receive each input.

20. The conversational agent according to claim 12, wherein at least one sub-dialogue unit is an orchestrator.
